(19) United States
(12) Patent Application Publication
(10) Pub. No.: US 2026/0047955 A1
(43) Pub. Date: Feb. 19, 2026

(54) CONDOM CATHETER

(71) Applicant: PUREWICK CORPORATION, Covington, GA (US)

(72) Inventors: Camille Rose Newton, Bonsall, CA (US); Raymond Newton, Bonsall, CA (US); Claire Gloeckner, Lilburn, GA (US)

(21) Appl. No.: 19/103,165

(22) PCT Filed: Aug. 22, 2022
(86) PCT No.: PCT/US2022/041085

§ 371 Date: Feb. 11, 2025

(51) Int. Cl. A61F 5/453; A61F 5/44

(52) U.S. Cl. CPC A61F 5/453; A61F 5/4401; A61F 5/4408

(57) ABSTRACT

Embodiments are directed to condom catheters, fluid collection systems including the same, and methods of using the same. An example condom catheter includes a fluid impermeable layer. In an embodiment, a condom catheter is disclosed. The condom catheter includes a fluid impermeable layer including an end region defining at least a portion of a chamber. The chamber is configured to receive at least a portion of a glans of a penis. The end region defines a fluid outlet. The fluid impermeable layer also includes at least one side wall extending from the distal end. At least a portion of an interior surface of the at least one side wall includes an adhesive. The at least one side wall defines an opening configured to receive a penis. The condom catheter also includes at least one porous material configured to be disposed in the chamber.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

Embodiments are directed to condom catheters, fluid collection systems including the same, and methods of using the same. An example condom catheter includes a fluid impermeable layer. In an embodiment, a condom catheter is disclosed. The condom catheter includes a fluid impermeable layer including an end region defining at least a portion of a chamber. The chamber is configured to receive at least a portion of a glans of a penis. The end region defines a fluid outlet. The fluid impermeable layer also includes at least one side wall extending from the distal end. The at least one side wall defines an opening configured to receive a penis. The at least one side wall is configured to switch between a collapsed state and an expanded state. The condom catheter also includes at least one porous material configured to be disposed in the chamber.

In an embodiment, a fluid collection system is disclosed. The system includes a condom catheter. The condom catheter includes a fluid impermeable layer including an end region defining at least a portion of a chamber. The chamber is configured to receive at least a portion of a glans of a penis. The end region defines a fluid outlet. The fluid impermeable layer also includes at least one side wall extending from the distal end. The at least one side wall defines an opening configured to receive a penis. The at least one side wall is configured to switch between a collapsed state and an expanded state. The condom catheter also includes at least one porous material configured to be disposed in the chamber. The system also includes a fluid storage container and a vacuum source. The chamber of the condom catheter, the fluid storage container, and the vacuum source are in fluid communication with each that, when one or more bodily fluids are present in the chamber, a suction provided from the vacuum source to the chamber of the condom catheter removes the one or more bodily fluids from the chamber and deposits the bodily fluids in the fluid storage container.

In an embodiment, a method to use a condom catheter is disclosed. The method includes positioning at least one porous material around at least a portion of a glans of a penis and positioning a fluid impermeable layer around the at least a portion of the glans of the penis such that the at least one porous material is positioned between at least a portion of the fluid impermeable layer and the penis. The fluid impermeable layer includes an end region defining at least a portion of the chamber. The chamber is configured to receive at least a portion of the glans of a penis. The end region defines a fluid outlet. The fluid impermeable layer also includes at least one side wall extending from the end region. The at least one side wall defines an opening configured to receive the penis. The at least one side wall is configured to switch between a collapsed state and an expanded state. The at least one porous material is disposed in the chamber at least after positioning the fluid impermeable layer around the at least a portion of the glans of the penis.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a condom catheter, according to an embodiment.

FIG. 1B is a cross-sectional view of the condom catheter taken along plane 1B-1B, according to an embodiment.

FIG. 1C is a cross-sectional view of the condom catheter taken along plane 1B-1B when the at least one side wall exhibits the expanded configuration, according to an embodiment.

FIG. 2 is a cross-sectional view of a condom catheter that includes a porous material including a plurality of layers, according to an embodiment.

FIG. 3 is a cross-sectional view of a condom catheter, according to an embodiment.

FIG. 4 is an exploded isometric view of a condom catheter, according to an embodiment.

FIG. 5 is an isometric view of a porous material including a slit, according to an embodiment.

FIG. 6 is a top plan view of a porous material exhibiting a different shape, according to an embodiment.

FIG. 7 is a cross-sectional view of a condom catheter, according to an embodiment.

FIG. 8 is a cross-sectional view of a condom catheter that includes an attachment structure configured to make removal of the condom catheter less painful, according to an embodiment.

FIG. 9 is a cross-sectional view of a condom catheter that includes an attachment structure that is distinct from the side wall, according to an embodiment.

FIG. 10 is a block diagram of a fluid collection system for fluid collection, according to an embodiment.

### DETAILED DESCRIPTION

Embodiments are directed to condom catheters, fluid collection systems including the same, and methods of using the same. An example condom catheter includes a fluid impermeable layer. The fluid impermeable layer includes an end region and at least one side wall extending from the end region. The end region defines at least a portion of a chamber that is configured to receive at least a portion of a glans of a penis. The end region also defines a fluid outlet. The side wall includes an adhesive on at least a portion of an interior surface thereof that is configured to attach the condom catheter to the penis. The side wall also defines an opening configured to receive the penis thereby allowing at least a portion of the glans of the penis to be disposed in the chamber. The condom catheter also includes at least one porous material that is disposed in the chamber or is configured to be disposed in the chamber.

During use, the at least one porous material is disposed around the penis. In an embodiment, disposing the at least one porous material around the penis also causes the fluid impermeable layer to be disposed around the penis when the at least one porous material is disposed in the chamber. In an embodiment, when the porous material is not disposed in the chamber, the fluid impermeable layer may be positioned around the porous material that is already disposed around the penis, thereby positioning the fluid impermeable layer around at least a portion of the penis. After positioning the fluid impermeable layer and the porous material around the penis, bodily fluids (e.g., urine, semen, etc.) may be discharged from the penis. The bodily fluids may be received into the porous material. The bodily fluids received into the porous material may flow towards and be removed from the chamber via the fluid outlet. For example, the condom catheter may include or be connected to a conduit. In such an example, the condom catheter may be in communication with a vacuum source via the conduit. The vacuum source may apply a suction to the chamber. The suction may cause the bodily fluids received by the porous material to flow towards the fluid outlet and be removed from the chamber via the conduit.

The condom catheters disclosed herein are an improvement over conventional condom catheters. Some conventional condom catheters only include a sleeve that is configured to fit over the penis (“sleeve condom catheters”). The sleeve condom catheters should snugly fit against the shaft of the penis to prevent bodily fluids from leaking from the sleeve condom catheter. For example, the sleeve condom catheters may form passageways between the sleeve and the shaft of the penis if the sleeve condom catheters are too large. However, the sleeve condom catheters may uncomfortably compress the penis and may push the penis out of the sleeve condom catheter if the sleeve condom catheter is too small. Thus, the fit of the sleeve condom catheter is critical to the functioning of the sleeve condom catheter. Other conventional condom catheters include a sleeve with an absorbent, hydrophilic material disposed therein such that the absorbent material contacts the urethral opening of the penis (“absorbent condom catheters”). Such absorbent condom catheters may receive the bodily fluids discharged from the penis thereby preventing or at least inhibiting pooling of the bodily fluids against the penis. However, the absorbent condom catheters still need to exhibit a snug fit against the penis to prevent the formation of gaps between the penis and the absorbent condom catheter through which bodily fluids that are not received into or leave the absorbent material may leak. Further, the absorbent material holds the bodily fluids against the skin of the penis about the urethral opening which may cause skin degradation. Still other condom catheters need a base to be secure around the penis.

The condom catheters disclosed herein are an improvement over at least the conventional condom catheters discussed above. For example, the condom catheters disclose herein include a porous material disposed therein. The porous material may extend along the sides of the condom catheter such that the porous material may be adjacent to the shaft of the penis. The porous material extending along the shaft of the penis is likely to receive bodily fluids that would otherwise leak between the penis and the fluid impermeable layer thereof. The porous material extending along the shaft of the penis also makes the size of the condom catheter less critical since the porous material may at least partially fill any gaps between the penis and the fluid impermeable layer when the condom catheter is too large and may provide cushioning if the condom catheter is too small. Further, unlike the absorbent material, the porous material of the condom catheters disclosed herein are configured to move the bodily fluids towards the fluid outlet thereby preventing the bodily fluids from remaining against the penis. Also, the condom catheters disclosed herein may be secured to the penis without using a base.

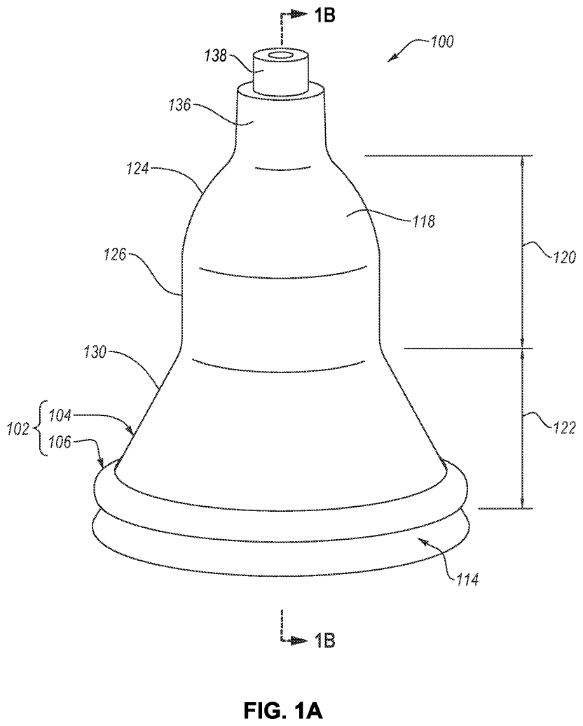

FIG. 1A is an isometric view of a condom catheter 100, according to an embodiment. FIG. 1B is a cross-sectional view of the condom catheter 100 taken along plane 1B-1B, according to an embodiment. The condom catheter 100 includes a fluid impermeable layer 102. The fluid impermeable layer 102 includes an end region 104 and at least one side wall 106 extending from the end region 104. The fluid impermeable layer 102 also defines a chamber 108, an opening 110 configured to allow a penis to be at least partially inserted into the chamber 108, and a fluid outlet 112. The fluid impermeable layer 102 also include at least one porous material 114 disposed in the chamber 108.

The fluid impermeable layer 102 at least partially defines a chamber 108 (e.g., interior region) and an opening 110. For example, the interior surface(s) 116 of the fluid impermeable layer 102 at least partially defines the chamber 108 within the condom catheter 100. The fluid impermeable layer 102 temporarily stores the bodily fluids in the chamber 108. The fluid impermeable layer 102 may also direct the bodily fluids towards the fluid outlet 112. The fluid impermeable layer 102 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, any other fluid impermeable material disclosed herein, or combinations thereof. As such, the fluid impermeable layer 102 substantially prevents the bodily fluids from passing through the fluid impermeable layer 102. In an example, the fluid impermeable layer 102 may be air permeable and fluid impermeable. In such an example, the fluid impermeable layer 102 may be formed of a hydrophobic material that defines a plurality of pores. At least one or more portions of at least an outer surface 118 of the fluid impermeable layer 102 may be formed from a soft and/or smooth material, thereby reducing chaffing. Portions of the interior surface 116 that may contact the penis may also be formed from a soft and/or smooth material to reduce chaffing.

As previously discussed, the fluid impermeable layer 102 includes the end region 104 and the side wall 106. The end region 104 may be configured to enclose, abut, or otherwise receive at least a portion of the glans of the penis. The side wall 106 is configured to enclose, abut, or otherwise receive the shaft of the penis. It is noted that the end region 104 may also receive a portion of the shaft of the penis and/or the side wall 106 may receive a portion of the glans of the penis.

In an embodiment, the end region 104 is distinguishable from the side wall 106 because the end region 104 exhibits a shape that is different than the side wall 106. The end region 104 and the side wall 106 exhibits different shapes because the end region 104 is configured to receive at least a portion of the glans (e.g., head) of the penis while the side wall 106 is configured to be adjacent to at least a portion of the shaft of the penis. As such, the end region 104 may exhibit a shape that generally corresponds to the shape of the glans of the penis and the side wall 106 may exhibit a shape that corresponds to the shaft of the penis. For instance, the end region 104 may exhibit a generally tapered shape (e.g., semi-spherical or conical shape) while the side wall 106 may exhibit a generally cylindrical shape.

In a particular example, the end region 104 includes a sump 120 and a bulge region 122. The sump 120 extends from the fluid outlet 112 to the bulge region 122 and the bulge region 122 may extend from sump 120 to the side wall 106. The sump 120 is configured to temporarily store bodily fluids therein until the bodily fluids are removed from the chamber 108 through the fluid outlet 112. The sump 120 may be at least partially occupied by the porous material 114 since the porous material 114 may facilitate storing the bodily fluids in the sump 120. In an embodiment, the sump 120 may be substantially entirely occupied (e.g., at least 80 volume percent occupied) by the porous material 114. Substantially occupying all of the sump 120 with the porous material 114 may prevent the formation of gaps in the sump 120 in which the bodily fluids may pool since the pooled bodily fluids may cause microbe growth and increase the likelihood that the bodily fluids leak from the sump 120. Substantially occupying the sump 120 with the porous material 114 may also facilitate flow of the bodily fluids towards the fluid outlet 112. For instance, the porous material 114 forms a plurality of interconnected passageways through which the bodily fluids may flow. The passageways of the porous material 114 may be sufficiently small that the bodily fluids in the porous material 114 may be attached together via hydrogen bonding. As such, removing some of the bodily fluids from the porous material 114 via the fluid outlet 112 pulls the rest of the bodily fluids towards the fluid outlet 112. In other words, the porous material 114 substantially occupying all of the sump 120 may prevent the formation of gaps that are too large for bodily fluids to pull themselves across via hydrogen bonding. The porous material 114 substantially occupying all of the sump 120 also prevents collapse of the sump 120 when a suction is applied to the chamber 108 since collapsing the sump 120 may inhibit flow of the bodily fluids therethrough.

The sump 120 may exhibit a generally tapered shape, such as a rounded or generally conical shape. For example, as illustrated, the sump 120 may include a first generally tapered surface 124 (e.g., conical surface) extending from the fluid outlet 112. The first generally tapered surface 124 increases the lateral dimension of the sump 120 and causes the bodily fluids to be funneled towards the fluid outlet 112. The sump 120 may also define a generally cylindrical surface 126 extending from the first generally tapered surface 124 to the bulge region 122. The generally cylindrical surface 126 may increase the volume of the sump 120 without increasing the lateral dimension of the sump 120. In other words, the generally cylindrical surface 126 allows greater flexibility in selecting the volume of the sump 120 and the angle that the first generally tapered surface 124 extends from the fluid outlet 112. It is noted that the generally cylindrical surface 126 may be omitted from the sump 120.

The bulge region 122 may include the porous material 114 disposed therein. However, the bulge region 122 is only partially occupied by the porous material 114. Only partially occupying the bulge region 122 with the porous material 114 allows the bulge region 122 to receive at least a portion of the glans of the penis. For example, the porous material 114 disposed in the bulge region 122 may define a cavity 128. The cavity 128 may include an unoccupied portion of the chamber 108 that may receive at least a portion of the glans of the penis and, optionally, at least a portion of the shaft of the penis. It is noted that the cavity 128 may extend partially into the sump 120. To form the cavity 128, the porous material 114 may be disposed adjacent to at least a portion of the interior surface 116 of the fluid impermeable layer 102 that forms the bulge region 122. In an example, the porous material 114 may be positioned adjacent to substantially all of the interior surface of the bulge region 122 which prevents formation of gaps between the penis disposed in the cavity 128 and the fluid impermeable layer 102 through which bodily fluids may leak.

In an embodiment, the bulge region 122 may include a generally tapered shape. The tapered shape of the bulge region 122 causes a maximum lateral dimension of the bulge region 122 to be larger than the maximum lateral dimension of the sump 120. The bulge region 122 may include at least one second generally tapered surface 130 that causes the bulge region 122 to exhibit the tapered shape. The second generally tapered surface 130 may extend from the sump 120 towards the side wall 106. The second generally tapered surface 130 may exhibit a rounded, generally conical, or other suitable shape. The second generally tapered surface 130 allows the bulge region 122 to better conform to the shape of the penis which, in turn, inhibits leakage from the condom catheter 100. The second generally tapered surface 130 may also cause the bulge region 122 to exhibit a cross-sectional shape and area that is large enough to receive the glans of the penis since, in some embodiments, the cross-sectional shape and area of the sump 120 may be too small to comfortably receive any significant portion of the glans of the penis. In an embodiment, not shown, the bulge region 122 may also include a second generally cylindrical surface. The second generally cylindrical surface may allow the bulge to better conform to the sides of the glans of the penis and/or the shaft of the penis when such portions of the penis are inserted into the end region 104. It is noted that the second generally cylindrical surface may be omitted from the bulge region 122.

It is noted that, in some embodiments, the sump 120 and/or the bulge region 122 may be omitted from the condom catheter 100.

As previously discussed, the side wall 106 is configured to receive at least a portion of the shaft of the penis. The length of the shaft of the penis may vary from individual to individual. The length of the shaft of the penis may also vary depending on whether the penis is erect or flaccid. To accommodate these variations in the lengths of the shaft, the side wall 106 may be configured to change a length thereof. Meanwhile, the end region 104 may not be configured to exhibit a length change since the glans of the penis exhibits less size variations than the shaft of the penis. In other words, the end region 104 may be distinguishable from the side wall 106 since the side wall 106 may change a length thereof while the length of the end region 104 remains relatively constant. It is noted that the lengths of the end region 104 and the side wall 106 is measured parallel to a longitudinal axis 132 of the condom catheter 100.

Since the side wall 106 is configured to change a length thereof, the side wall 106 may exhibit a collapsed state and an expanded state. FIGS. 1B and 1C are cross-sectional views of the condom catheter 100 when the side wall 106 exhibits the collapsed and expanded states, respectively. As used herein, the collapsed state of the side wall 106 refers to the state of the side wall 106 when the side wall 106 exhibits the minimum length thereof and the expanded state of the side wall 106 refers to the state of the side wall 106 when the side wall 106 exhibits the maximum length thereof. The side wall 106 may also exhibit at least one intermediate state between the collapsed state and the expanded state. The intermediate state refers to the state of the side wall 106 when the side wall 106 exhibits a length between the minimum length of the collapsed state and the maximum length of the expanded state. The collapsed, expanded, and intermediate states of the side wall 106 allows the side wall 106 to selectively exhibit a length for each penis. In an embodiment, the condom catheter 100 may be provided with the side wall 106 in the collapsed state since it may be easier for a user of the condom catheter 100 to increase the length of the side wall 106 than decrease the length of the side wall 106 and to minimize the size of the condom catheter 100 for shipping, handling, and packaging. However, it is noted that the condom catheter 100 may be provided with the side wall 106 in the intermediate or expanded states thereof.

The difference in the length of the side wall 106 between the collapsed state thereof and the expanded state thereof may be about 2 cm or greater, about 4 cm or greater, about 6 cm or greater, about 8 cm or greater, about 10 cm or greater, about 12.5 cm or greater, about 15 cm or greater, about 17.5 cm or greater, about 20 cm or greater, about 22.5 cm or greater, about 25 cm or greater, or in ranges of about 2 cm to about 6 cm, about 4 cm to about 8 cm, about 6 cm to about 10 cm, about 8 cm to about 12.5 cm, about 10 cm to about 15 cm, about 12.5 cm to about 17.5 cm, about 15 cm to about 20 cm, about 17.5 cm to about 22.5 cm, or about 20 cm to about 25 cm. The difference in the length of the side wall 106 between the collapsed and expanded states thereof may be selected based on the size of penis and/or the ranges in sizes of the penises that the condom catheter 100 is configured to be used with. The difference in the length of the side wall 106 between the collapsed and expanded states thereby may be selected based on whether the side wall 106 is configured to extend down the whole length of the shaft of the penis or only a portion of the length of the penis. the difference in the length of the side wall 106 between the collapsed and expanded states may be selected based on the length needed on a particular patient at a particular time.

In an embodiment, as illustrated, the side wall 106 is completely rolled up when the side wall 106 is in the collapsed state. In such an embodiment, the side wall 106 is switched from the collapsed state to the intermediate state, the collapsed state to the expanded state, the intermediate state to the expanded state, or between different intermediate states by unrolling the side wall 106. The side wall 106 is also switched from the expanded state to the intermediate state, the expanded state to the collapsed state, the intermediate state to the collapsed state, or between different intermediate states by rolling up the side wall 106. In an embodiment, the side wall 106 is folded up when the side wall 106 is in the collapsed state. In such an embodiment, the side wall 106 is switched from the collapsed state to the intermediate state, the collapsed state to the expanded state, the intermediate state to the expanded state, or between different intermediate states by unfolding the side wall 106. The side wall 106 is also switched from the expanded state to the intermediate state, the expanded state to the collapsed state, the intermediate state to the collapsed state, or between different intermediate states by folding up the side wall 106. It is noted that the side wall 106 may be in the collapsed state using techniques other than rolling or folding the side wall 106, such as crunching, compressing, or otherwise changing the length of the side wall 106.

In an embodiment, the end region 104 may be distinguishable from the side wall 106 because the end region 104 exhibits a rigidity that is greater than the side wall 106. The side wall 106 may be formed of a relatively flexible material relative to the end region 104 to facilitate switching the side wall 106 between the collapsed, expanded, and intermediates states thereof. Meanwhile, the end region 104 may be formed of a relatively rigid material relative to the side wall 106 since the length of the end region 104 may remain substantially constant and to prevent collapse of the end region 104. In an example, the end region 104 may be formed from a material exhibiting a Young's modulus (e.g., modulus of elasticity) that is greater than a material that forms the side wall 106 such that the end region 104 is more rigid than the side wall 106. In such an example, the side wall 106 may need to be attached to the end region 104 using an adhesive, ultrasonic welding, radio frequency welding, or any other suitable technique. In an example, the end region 104 may exhibit a thickness that is greater than the side wall 106 thereby causing the end region 104 to exhibit a rigidity that is greater than the side wall 106.

The side wall 106 may exhibit a maximum lateral dimension (e.g., diameter) when in the expanded configuration that is about 2 cm or greater, about 2.5 cm or greater, about 3 cm or greater, about 3.5 cm or greater, about 4 cm or greater, about 4.5 cm or greater, about 5 cm or greater, about 5.5 cm or greater, about 6 cm or greater, about 6.5 cm or greater, about 7 cm or greater, about 7.5 cm or greater, about 8 cm or greater, or in ranges of about 2 cm to about 3 cm, about 2.5 cm to about 3.5 cm, about 3 cm to about 4 cm, about 3.5 cm to about 4.5 cm, about 4 cm to about 5 cm, about 4.5 cm to about 5.5 cm, about 5 cm to about 6 cm, about 5.5 cm to about 6.5 cm, about 6 cm to about 7 cm, about 6.5 cm to about 7.5 cm, or about 7 cm to about 8 cm. Conventional condom catheter 100 generally exhibit a maximum lateral dimension that is less than 4.5 cm to prevent the formation of gaps between the conventional condom catheter 100s and the penis. However, the side wall 106 when in the expanded configuration is greater than 4.5 cm because the porous material 114 may be adjacent to at least a portion of the side wall 106. The porous material 114 prevents or at least inhibits the formation of gaps between the condom catheter 100 and the penis through which bodily fluids may leak.

The fluid impermeable layer 102 may include an adhesive 134 (shown schematically with a bolded line) disposed on at least a portion of the interior surface 116 thereof. In an example, the adhesive 134 may be used to attach the fluid impermeable layer 102 to the porous material 114 thereby securing the porous material 114 to fluid impermeable layer 102. In an example, the adhesive 134 may be used to attach the fluid impermeable layer 102 to the penis, thereby strongly securing the condom catheter 100 to the penis and providing a seal that may inhibit leakage of bodily fluids from the condom catheter 100. In an example, the adhesive 134 may be disposed on at least a portion and, preferably, substantially all of the interior surfaces of the side wall 106. Disposing the adhesive 134 on all of the side wall 106 ensures that whichever portions of the side wall 106 that are adjacent to the porous material 114 and/or the penis has an adhesive 134 to secure the side wall 106 to the porous material 114 and/or the penis regardless of which state the side wall 106 is in. In an embodiment, the adhesive 134 may include a hydrocolloid adhesive 134 since the hydrocolloid adhesive 134 may wick bodily fluids towards the fluid outlet 112. In an embodiment, the adhesive 134 may include a hydrogel or any other suitable adhesive 134.

In an embodiment, the condom catheter 100 may include a connector 136 extending from the fluid outlet 112. For example, the fluid outlet 112 may be a hole extending through the fluid impermeable layer 102 and the connector 136 may extend from the fluid outlet 112. The connector 136 is configured to receive a conduit 138 and have the conduit 138 secured thereto. As such, the connector 136 allows the conduit 138 to be in fluid communication with the chamber 108 through the fluid outlet 112. In an embodiment, as shown, the connector 136 is integrally formed with the fluid impermeable layer 102 (e.g., the fluid impermeable layer 102 and the connector 136 exhibit single piece construction). In an embodiment, the connector 136 is distinct from and attached to the fluid impermeable layer 102.

As previously discussed, the condom catheter 100 includes the porous material 114 in the chamber 108. The porous material 114 is configured to receive the bodily fluids discharged from the urethral opening of the penis and move the bodily fluids towards the fluid outlet 112. Thus, the porous material 114 helps remove the bodily fluids from the chamber 108 and helps keep the penis dry to inhibit degradation of the skin of the penis. The porous material 114 also prevents or at least inhibits leakage of the bodily fluid from the condom catheter 100. The porous material 114 further fills in gaps when the condom catheter 100 is too large. In an embodiment, as shown, the porous material 114 includes a single structure one or more layers. In an embodiment, the porous material 114 includes a plurality of distinct structures, such as a first structure disposed in the end region 104 and a second structure disposed adjacent to the side wall 106.

In an embodiment, the porous material 114 may be configured to wick any bodily fluids away from the opening 110, thereby preventing the bodily fluids from escaping the chamber 108. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the porous material 114. Put another way, substantially no absorption or solubility of the bodily fluids into the porous material 114 may take place after the porous material 114 is exposed to the bodily fluids and removed from the bodily fluids for a time. While no absorption or solubility is desired, the term “substantially no absorption” may allow for nominal amounts of absorption and/or solubility of the bodily fluids into the porous material 114 (e.g., absorbency), such as less than about 30 wt % of the dry weight of the porous material 114, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the porous material 114. The porous material 114 may also wick the bodily fluids generally towards an interior of the chamber 108, as discussed in more detail below. In an embodiment, the porous material 114 may include at least one absorbent or adsorbent material.

The porous material 114 may include any material that may wick, absorb, adsorb, or otherwise allow fluid transport of the bodily fluids. In an example, the porous material 114 may include fabric, such as a gauze (e.g., a silk, linen, or cotton gauze), another soft fabric, another smooth fabric, or a nonwoven material. Forming the porous material 114 from gauze, soft fabric, and/or smooth fabric may reduce chaffing caused by the condom catheter 100. In an example, the porous material 114 may include a porous polymer (e.g., nylon, polyester, polyurethane, polyethylene, polypropylene, etc.) structure or an open cell foam, such as spun nylon fiber. In an example, the porous material 114 may be formed from a natural material, such as cotton, wool, silk, or combinations thereof. In such examples, the porous material 114 may have a coating to prevent or limit absorption of fluid into the material, such as a water repellent coating.

In an embodiment, at least a portion of the porous material 114 may be hydrophobic. The porous material 114 may be hydrophobic when the porous material 114 exhibits a contact angle with water (a major constituent of bodily fluids) that is greater than about 90°, such as in ranges of about 90° to about 120°, about 105° to about 135°, about 120° to about 150°, about 135° to about 175°, or about 150° to about 180°. The hydrophobicity of the porous material 114 may limit absorption, adsorption, and solubility of the bodily fluids in the porous material 114, thereby decreasing the amount of bodily fluids held in the porous material 114. In an embodiment, at least a portion of the porous material 114 is hydrophobic or hydrophilic.

In an embodiment, the porous material 114 is initially disposed in the chamber 108. In an embodiment, the porous material 114 is not initially disposed in the chamber 108 and, instead, is configured to be disposed in the chamber 108. In such an embodiment, the porous material 114 is initially separate and spaced from the fluid impermeable layer 102. When the porous material 114 is not initially disposed in the chamber 108, the condom catheter 100 may be formed by disposing the porous material 114 around at least a portion of the penis and then the fluid impermeable layer 102 is disposed over the porous material 114. Alternatively, the porous material 114 may be disposed in the fluid impermeable layer 102 before positioning the porous material 114 around the penis to form the condom catheter 100 and then disposing the formed condom catheter 100 around at least a portion of the penis. Initially disposing the porous material 114 in the chamber 108 makes using the condom catheter 100 easier since the condom catheter 100 does not need to be assembled and prevents a user of the condom catheter 100 from incorrectly assembling the condom catheter 100 (e.g., incorrectly assembling the condom catheter 100 may form wrinkles in the porous material 114 through which bodily fluids may leak or pool). However, not disposing the porous material 114 initially in the fluid impermeable layer 102 provides several benefits. For example, it may be easier to cut or otherwise cause the porous material 114 to exhibit a length that corresponds to the penis if the porous material 114 is not disposed in the fluid impermeable layer 102. Also, not disposing the porous material 114 in the fluid impermeable layer 102 allows the porous material 114 to retrofit conventional condom catheters to form the condom catheter 100 by disposing the porous material 114 in the conventional condom catheters. Additionally, not disposing the porous material 114 in the fluid impermeable layer 102 allows the porous material 114 to be specifically selected for each penis. For instance, a relatively thin porous material 114 may be selected if the width of the penis is relatively wide to prevent or at least inhibit the condom catheter 100 uncomfortably compressing the penis or a relatively thick porous material 114 may be selected if the width of the penis is relatively narrow to prevent or at least inhibit gaps between the penis and the porous material 114 through which bodily fluids may leak. It is noted that the relatively thick porous material 114 may facilitate flow of the bodily fluids therethrough better than the relatively think porous material 114.

Regardless if the porous material 114 is or is not initially disposed in the chamber 108, the porous material 114 may exhibit a size and shape configured to be disposed in the chamber 108. For example, an exterior surface of the porous material 114 may be configured to exhibit a size and shape that corresponds to at least a portion of the interior surface 116 of the fluid impermeable layer 102. As such, disposing the porous material 114 in the chamber 108 prevents the formation of gaps between the fluid impermeable layer 102 and the porous material 114. The porous material 114 may also exhibit a size, shape, and volume that is configured to allow the porous material 114 to substantially completely occupy the sump 120.

The porous material 114 may be and/or is configured to be positioned adjacent to at least a portion of the interior surface 116 of the side wall 106. Disposing the porous material 114 adjacent to the side wall 106 allows the porous material 114 to remove bodily fluids (e.g., sweat) from the shaft of the penis thereby keeping the shaft of the penis dry, provide cushioning to the shaft of the penis, allow variations in the size of the penis, and inhibit leakage of the bodily fluids along the shaft of the penis. In an example, the porous material 114 may be and/or is configured to be positioned adjacent to about 10% or more of the surface area of the interior surface 116 of the side wall 106, such as about 20% or more, about 30% or more, about 40% or more, about 50% or more, about 60% or more, about 70% or more, about 80% or more, about 90% or more, about 95% or more, or in ranges of about 10% to about 30%, about 20% to about 40%, about 30% to about 50%, about 40% to about 60%, about 50% to about 70%, about 60% to about 80%, about 70% to about 90%, or about 85% to about 95% of the surface area of the interior surface 116 of the side wall 106. In an example, the porous material 114 may be and/or is configured to be positioned adjacent to along 10% or more of the length of the side wall 106, such as about 20% or more, about 30% or more, about 40% or more, about 50% or more, about 60% or more, about 70% or more, about 80% or more, about 90% or more, about 95% or more, or in ranges of about 10% to about 30%, about 20% to about 40%, about 30% to about 50%, about 40% to about 60%, about 50% to about 70%, about 60% to about 80%, about 70% to about 90%, or about 85% to about 95% of the length of the side wall 106. In an embodiment, the porous material 114 is not disposed adjacent to at least a portion of the side wall 106 thereby allowing the side wall 106 to be disposed adjacent to and attached to the shaft of the penis.

Similar to the side wall 106, the porous material 114 may be configured to change a length thereof thereby allowing the porous material 114 to be attached to the side wall 106. As such, the porous material 114 may exhibit a collapsed state and an expanded state. FIGS. 1B and 1C are cross-sectional views of the condom catheter 100 when the porous material 114 exhibits the collapsed and expanded states, respectively. As used herein, the collapsed state and the expanded state of the porous material 114 refers to the state of the porous material 114 when the porous material 114 exhibits the minimum length thereof and the maximum length thereof, respectively. The porous material 114 may also exhibit at least one intermediate state between the collapsed state and the expanded state. The intermediate state refers to the state of the porous material 114 when the porous material 114 exhibits a length between the minimum length of the collapsed state and the maximum length of the expanded state. The collapsed, expanded, and intermediate states of the porous material 114 allows the porous material 114 to selectively exhibit a length for each penis and allows the porous material 114 to be disposed adjacent to a selected surface area and/or length of the side wall 106 depending on whether the side wall 106 exhibits the collapsed, expanded, or intermediate state thereof. In an embodiment, the condom catheter 100 may be provided with the porous material 114 in the collapsed state since it may be easier for a user of the condom catheter 100 to increase the length of the porous material 114 than decrease the length of the porous material 114 and to minimize the size of the condom catheter 100 for shipping, handling, and packaging.

The difference in the length of the porous material 114 between the collapsed state thereof and the expanded state thereof may be about 2 cm or greater, about 4 cm or greater, about 6 cm or greater, about 8 cm or greater, about 10 cm or greater, about 12.5 cm or greater, about 15 cm or greater, about 17.5 cm or greater, about 20 cm or greater, about 22.5 cm or greater, about 25 cm or greater, or in ranges of about 2 cm to about 6 cm, about 4 cm to about 8 cm, about 6 cm to about 10 cm, about 8 cm to about 12.5 cm, about 10 cm to about 15 cm, about 12.5 cm to about 17.5 cm, about 15 cm to about 20 cm, about 17.5 cm to about 22.5 cm, or about 20 cm to about 25 cm. The difference in the length of the porous material 114 between the collapsed and expanded states thereof may be selected for the same reasons as the difference in the length of the side wall 106, as previously discussed.

In an embodiment, as illustrated, the porous material 114 is completely rolled up when the side wall 106 is in the collapsed state. In such an embodiment, the porous material 114 is switched between states by unrolling or rolling the side wall 106. In an embodiment, the porous material 114 is folded up when the porous material 114 is in the collapsed state. In such an embodiment, the porous material 114 is switched between the states thereof by unfolding or folding the porous material 114. It is noted that the porous material 114 may be in the collapsed state using techniques other than rolling or folding the side wall 106, such as crunching, compressing, or otherwise changing the length of the porous material 114.

In an embodiment, not shown, the porous material 114 is initially attached to the side wall 106. In such an embodiment, the porous material 114 may be in the same state as the side wall 106 and switches between states when the side wall 106 switches between states. In an embodiment, as shown in FIGS. 1A and 1B, the porous material 114 is not initially attached to the side wall 106. In such an embodiment, the porous material 114 may be switched between states independently from the side wall 106 at least until the porous material 114 is attached to the side wall 106. For example, the porous material 114 may be switched from the collapsed state thereof to the expanded state thereof while the side wall 106 remains in the collapsed state thereof. Allowing the porous material 114 to be switched independently of the side wall 106 may facilitate operation of the condom catheter 100. For instance, not attaching the porous material 114 to the side wall 106 may make it easier to cut the porous material 114 or dispose the porous material 114 against a selected portion of the shaft of the penis than if the porous material 114 is attached to the porous material 114. It is noted that switching the side wall 106 from the collapsed state to an intermediate or expanded state thereof may cause the side wall 106 to become attached to the porous material 114.

In an embodiment, the porous material 114 may extend through or be configured to extend through the fluid outlet 112. In such an embodiment, the porous material 114 may be disposed in at least a portion of the connector 136 and, optionally, into at least a portion of the conduit 138. Disposing the porous material 114 into the connector 136 and/or into the conduit 138 may inhibit collapse of the connector 136 and/or the conduit 138 since collapsing the connector 136 or the conduit 138 may prevent or at least inhibit removal of bodily fluids from the chamber 108. For example, the porous material 114 in the connector 136 and/or the conduit 138 may prevent collapse thereof when an object is disposed thereon or when the connector 136 and/or conduit 138 is bent in such a manner that, without the porous material 114, the connector 136 and/or the conduit 138 would kink. The porous material 114 disposed in the connector 136 and/or the conduit 138 may also facilitate removal of some the bodily fluids from the chamber 108 via capillary action even when a suction is not applied to the chamber 108.

In an embodiment, not shown, the porous material 114 and the cavity 128 may not substantially completely fill the portions of the chamber 108. In such an embodiment, the condom catheter 100 includes the reservoir disposed in the chamber 108 between the porous material 114 and the fluid impermeable layer 102. The reservoir is a substantially unoccupied portion of the chamber 108 between the porous material 114 and the fluid impermeable layer 102. The bodily fluids that are in the chamber 108 may flow through the porous material 114 to the reservoir. The reservoir may temporarily retain of the bodily fluids therein.

As previously discussed, the condom catheter 100 may include a conduit 138. The conduit 138 may be used to remove the bodily fluids from the chamber 108. The conduit 138 is configured to fluidly couple (e.g., directly or indirectly) the vacuum source (not shown) to the chamber 108. As the vacuum source applies a suction in the conduit 138, the bodily fluids in the chamber 108 (e.g., at the end region 104) may be drawn into the conduit 138. In some examples, the conduit 138 may be frosted or opaque (e.g., black) to obscure visibility of the bodily fluids therein.

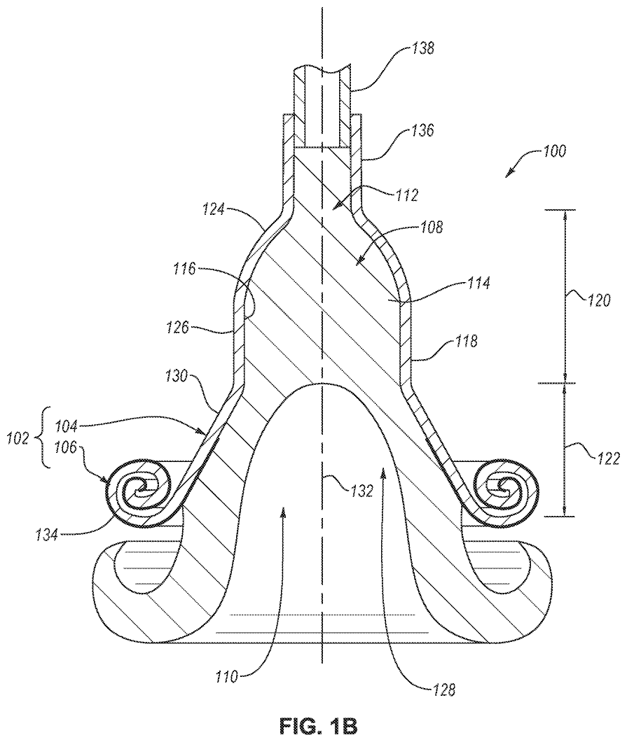

The porous material 114 of the condom catheter 100 includes a single material. However, the condom catheters disclosed herein may include a plurality of materials/layers. For example, FIG. 2 is a cross-sectional view of a condom catheter 200 that includes a porous material 214 including a plurality of layers, according to an embodiment. Except as otherwise disclosed herein, the condom catheter 200 may be the same as or substantially similar to any of the condom catheters 200 disclosed herein. For example, the condom catheter 200 includes a fluid impermeable layer 202 including an end region 204 and at least one side wall 206. The condom catheter 200 also includes the porous material 214 that is disposed in or configured to be disposed in a chamber 208 defined by the fluid impermeable layer 202.

The porous material 214 may include the fluid permeable outer layer 240 disposed in the chamber 208. The fluid permeable outer layer 240 may be composed to wick the bodily fluids away from the penis, thereby preventing the bodily fluids from escaping the chamber 208 or remaining against the penis. In an embodiment, the fluid permeable outer layer 240 may include any material that may wick the bodily fluids. For example, the fluid permeable outer layer 240 may include fabric, such as a gauze (e.g., a silk, linen, or cotton gauze), another soft fabric, another smooth fabric, a nonwoven material, or any of the other porous materials disclosed herein.

The porous material 214 may include the fluid permeable inner layer 242 disposed in the chamber 208. The fluid permeable inner layer 242 is configured to support the fluid permeable outer layer 240 since the fluid permeable outer layer 240 may be formed from a relatively foldable, flimsy, or otherwise easily deformable material. For example, the fluid permeable inner layer 242 may be positioned such that the fluid permeable outer layer 240 is disposed between the fluid permeable inner layer 242 and the fluid impermeable layer 202. As such, the fluid permeable inner layer 242 may support and maintain the position of the fluid permeable outer layer 240. The fluid permeable inner layer 242 may include any material that may wick, absorb, adsorb, or otherwise allow fluid transport of the bodily fluids, such as any of the fluid permeable membrane materials disclosed herein above. For example, the fluid permeable membrane material(s) may be utilized in a more dense or rigid form than in the fluid permeable outer layer 240 when used as the fluid permeable inner layer 242. The fluid permeable inner layer 242 may be formed from any fluid permeable material that is less deformable than the fluid permeable outer layer 240. For example, the fluid permeable inner layer 242 may include a porous polymer (e.g., nylon, polyester, polyurethane, polyethylene, polypropylene, etc.) structure or an open cell foam, such as spun nylon fiber. In some examples, the fluid permeable inner layer 242 may include a nonwoven material. In some examples, the fluid permeable inner layer 242 may be formed from a natural material, such as cotton, wool, silk, or combinations thereof. In such examples, the material may have a coating to prevent or limit absorption of fluid into the material, such as a water repellent coating. In some examples, the fluid permeable inner layer 242 may be formed from fabric, felt, gauze, or combinations thereof.

In an embodiment, the fluid permeable inner layer 242 is more hydrophobic (e.g., exhibits a larger contact angle with water) than the fluid permeable outer layer 240. The lower hydrophobicity of the fluid permeable outer layer 240 may help the porous material 214 receive the bodily fluids from the urethral opening while the hydrophobicity of the fluid permeable inner layer 242 limits the bodily fluids that are retained in the porous material 214.

The fluid permeable inner layer 242 includes a terminal edge 244 which is the portion of the fluid permeable inner layer 242 that is furthest spaced from the fluid outlet 212 and/or closest to the opening 210. In an embodiment, the fluid permeable outer layer 240 may extend around and cover the terminal edge 244 of the fluid permeable inner layer 242. In other words, the fluid permeable outer layer 240 may extend around the terminal edge 244 such that the fluid permeable outer layer 240 terminates at the fluid impermeable layer 202. As previously discussed, the fluid permeable outer layer 240 receives the bodily fluids more readily than the fluid permeable inner layer 242. As such, disposing the fluid permeable outer layer 240 around the terminal edge 244 better pulls bodily fluids into the porous material 214 that would otherwise pool near the terminal edge 244 and keeps more of the bodily fluids that would otherwise flow out of the fluid permeable inner layer 242 within the porous material 214.

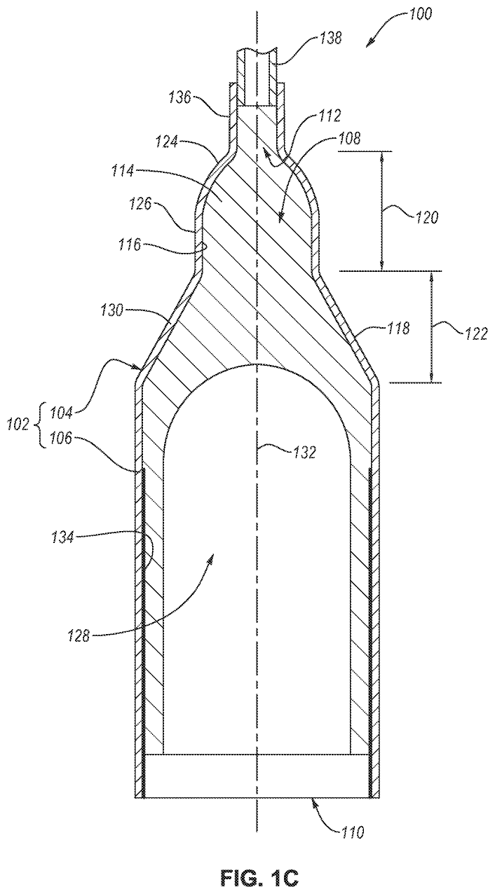

FIG. 3 is a cross-sectional view of a condom catheter 300, according to an embodiment. Except as otherwise disclosed herein, the condom catheter 300 is the same as or substantially similar to any of the condom catheters disclosed herein. For example, the condom catheter 300 may include a fluid impermeable layer 302 defining a chamber 308 and a fluid outlet 312. The fluid impermeable layer 302 may include an end region 304 and at least one side wall 306. The condom catheter 300 also includes at least one porous material 314.

The fluid impermeable layer 302 defines one or more perforations 346 extending therethrough. The perforations 346 allow air to flow from an exterior of the fluid impermeable layer 302 and into the chamber 308. The air flow through the perforations 346 allows air to flow from the perforations 346 to the fluid outlet 312 thereby pushing the bodily fluids in the chamber 308 towards the fluid outlet 312. In other words, the perforations 346 facilitate removal of the bodily fluids from the chamber 308. The air flow from the perforations 346 to the fluid outlet 312 also promotes evaporation of any bodily fluids that remain in the chamber 308 thereby making the penis drier. Further, the perforations 346 allows the chamber 308 to substantially remain at ambient pressure even when a suction is provided to the chamber 308 thereby preventing a suction injury to the penis (e.g., a hickey). It is noted that, without the perforations 346, there may be minimal air flow through the chamber 308 when the side walls 306 are adhesively attached to the shaft of the penis.

The perforations 346 may exhibit any suitable size. In an embodiment, the perforations 346 may be microscopic perforations exhibiting a maximum lateral dimension that is about 900 μm or less, about 750 μm or less, about 500 μm or less, about 250 μm or less, about 100 μm or less, about 50 μm or less, or in ranges of about 50 μm to about 250 μm, about 100 μm to about 500 μm, about 250 μm to about 750 μm, or about 500 μm to about 900 μm. The microscopic perforations may substantially prevent bodily fluids leaking through the perforations 346 due to the contact angle between the bodily fluids and the fluid impermeable layer 302. In an embodiment, the perforations 346 may be macroscopic perforations exhibiting a maximum lateral dimension that is about 1 mm or greater, about 1.5 mm or greater, about 2 mm or greater, about 2.5 mm or greater, about 3 mm or greater, about 4 mm or greater, about 5 mm or greater or in ranges of about 1 mm to about 2 mm, about 1.5 mm to about 2.5 mm, about 2 mm to about 3 mm, about 2.5 mm to about 4 mm, or about 3 mm to about 5 mm. The macroscopic perforations allow more air to flow therethrough without tearing the fluid impermeable layer 302 compared to the microscopic perforations which, in turn, allows stronger suctions to be applied to the chamber 308. The stronger suction allows for more bodily fluids to be removed from the chamber 308. It has been found that bodily fluids are still unlikely to leak through the perforations 346 when the perforations 346 are macroscopic perforations because the porous material 314 preferentially retains the bodily fluids and the air flow through the perforations 346 caused by the suction applied to the chamber 308 prevent bodily fluids leaking through the perforations 346. The relatively large maximum lateral dimensions of the macroscopic perforations allows the macroscopic perforations to include one or more slits (e.g., a single slit or a plurality of slits). The slits may inhibit leakage through the perforations 346 when no suction is provided to the chamber 308 because the slits allow the macroscopic perforations to remain substantially closed when no air is flowing therethrough. It is noted that the relatively small size of the microscopic perforations makes forming such slits difficult or irrelevant. It is noted that the perforations 346 may exhibit shapes other than slits, such as generally circular holes.

The perforations 346 may be formed in any portion of the chamber. In an embodiment, the perforations 346 are not formed in the side wall 306 since, during use, the side wall 306 are more likely to be covered by the skin of the individual (i.e., the skin is more likely to obstruct any perforations formed in the side wall 106) than the end region 104. In an embodiment, the perforations 346 are formed in a portion of the end region 304 that is adjacent or proximate to (e.g., within about 1 cm of, within about 5 mm of, or within 2 mm of) the side wall 306. In such an embodiment, the perforations 346 allow the air to flow through substantially all of the portions of the chamber 308 that are defined by the end region 304 thereby facilitating removal of the bodily fluids from such portions of the chamber 308.

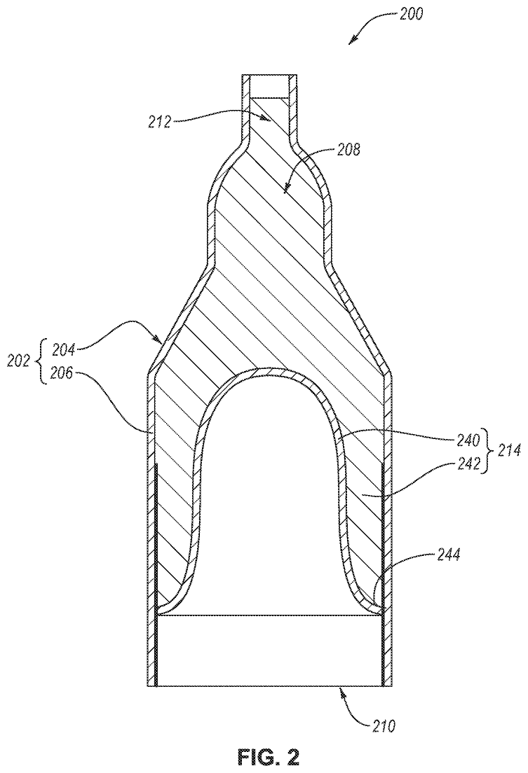

FIG. 4 is an exploded isometric view of a condom catheter 400, according to an embodiment. Except as otherwise disclosed herein, the condom catheter 400 is the same as or substantially similar to any of the condom catheters disclosed herein. For example, the condom catheter 400 includes a fluid impermeable layer 402 including an end region 404 and at least one side wall 406. The fluid impermeable layer 402 defines a chamber (not shown, obscured). The condom catheter 400 also includes at least one porous material 414.

As previously discussed, the porous material 414 is configured to be at least partially disposed in the chamber. For example, the porous material 414 may exhibit a shape that generally corresponds to the shape of the chamber. In the illustrated embodiment, the porous material 414 includes a first portion 448 that exhibits a shape that corresponds to the shape of the end region 404 thereby allowing the first portion 448 to be positioned adjacent to the interior surface(s) of the end region 404. The porous material 414 may also include a second portion 450 that exhibits a shape that corresponds to the shape of the side wall 406 when the side wall is in the expanded configuration thereby allowing the second portion 450 be positioned adjacent to the interior surface(s) of the side wall 406. The second portion 450 may be provided in a collapsed configuration (as shown in FIG. 1A) or an expanded configuration (as shown in FIG. 4). It is noted that, in some embodiments, the first portion 448 and/or second portion 450 of the porous material 414 may exhibit a shape that does not correspond to the shape of the end region 404 and/or the side wall 406, respectively.

As previously discussed, the porous material 414 may be initially disposed in the chamber (as shown in FIGS. 1A-1C) or may not be initially disposed in the chamber (as shown in FIG. 4). When the porous material 414 is not initially disposed in the chamber, the condom catheter 400 may be formed by disposing the porous material 414 around at least a portion of the penis and then the fluid impermeable layer 402 is disposed over the porous material 414. Alternatively, the porous material 414 may be disposed in the fluid impermeable layer 402 to form the condom catheter 400 and then disposing the formed condom catheter 400 around at least a portion of the penis.

In an embodiment, the porous material 414 may include an open end 452 and a closed end 454 opposite the open end 452. The open end 452 allows the porous material 414 to receive the penis. For example, the open end 452 allows the penis to be disposed in the cavity (e.g., cavity 128 shown in FIG. 1C) defined by the porous material 414. The closed end 454 is disposed adjacent to the glans of the penis and, in particular, the urethral opening of the penis. As such, the closed end 454 initially receives and blunts the stream of bodily fluids discharged from the urethral opening.

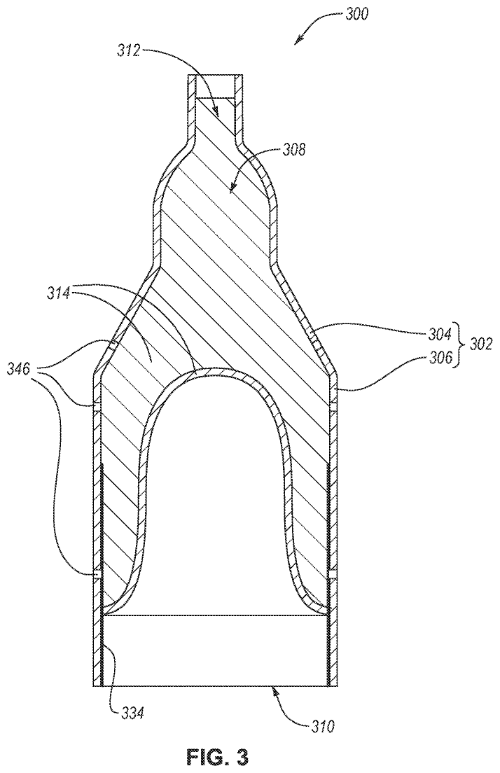

The porous materials disclosed herein may exhibit shapes other than the shape of the porous material 414 illustrated in FIG. 4. For example, FIG. 5 is an isometric view of a porous material 514 including a slit 556, according to an embodiment. Except as otherwise disclosed herein, the porous material 514 may be the same as or substantially similar to any of the porous materials disclosed herein. The porous material 514 may be used in any of the condom catheters disclosed herein.

The porous material 514 defines at least one slit 556. The slit 556 extends from the open end 552 of the porous material 514 towards the closed end 554. The slit 556 facilitates disposing the porous material 514 around the penis. For example, without the slit 556, the porous material 514 may be disposed around the penis by inserting the penis into the open end 552 of the porous material 514 and then sliding the penis through the porous material 514. However, due to friction, sliding the penis through the porous material 514 may be difficult and/or uncomfortably rub against the penis. The slit 556 allows the penis to be inserted through the slit 556 in addition to the open end 552 thereby decreasing or eliminating the need to slide the penis through the porous material 514. The slit 556 also makes it easier to shape and fold the porous material 514 around the penis.

The slit 556 also makes it easier to cut the porous material 514 when the porous material 514 is disposed on the penis prior to cutting the porous material 514. For example, it may be easier to accurately determine the needed length of the porous material 514 by first disposing the porous material 514 on the penis since it precludes the need to measure the penis with a measuring device. With the slit 556, the excess portions of the porous material 514 (i.e., the portions of the porous material that need to be cut) will flare away from the penis. As such, the portions of the porous material 514 that need to be cut are spaced from the penis which would not be the case if the porous material 514 did not include the slit 556.

In an embodiment, the slit 556 extends from the open end 552 to a location that is spaced from the closed end 554 of the porous material 514. In such an embodiment, the closed end 554 is able to receive and blunt the bodily fluids discharged from the urethral opening of the penis. However, the penis may need to be slide along a portion of the length of the porous material 514 to correctly position the penis in the porous material 514. In an embodiment, the slit 556 extends from the open end 552 to the closed end 554. In such an embodiment, the penis may be correctly positioned in the porous material 514 without sliding the penis within the porous material 514. However, the closed end 554 may be unable to initially receive and blunt urine discharged from the urethral opening into the portions of the slit 556 extending into the closed end 554 which may make it more difficult for the porous material 514 to receive the bodily fluids.

It is noted that any of the fluid impermeable layers disclosed herein may define a slit instead of or in addition to the porous material 514 defining the slit 556. In an embodiment, the slit defined in the fluid impermeable layer may be aligned with the slit 556 defined by the porous material 514.

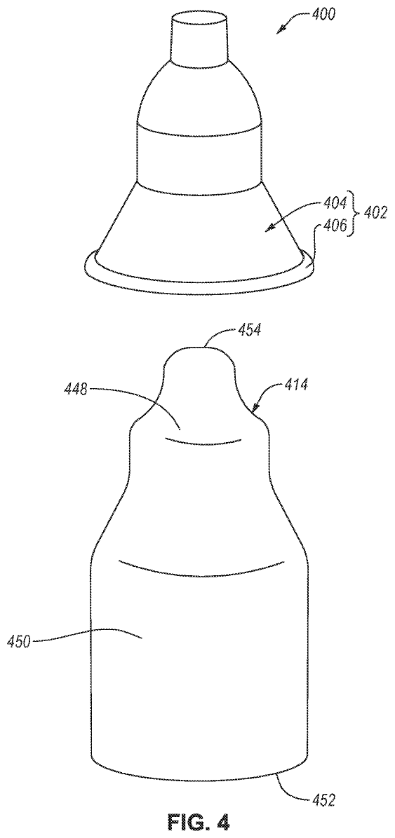

FIG. 6 is a top plan view of a porous material 614 exhibiting a different shape, according to an embodiment. Except as otherwise disclosed herein, the porous material 614 may be the same as or substantially similar to any of the porous materials disclosed herein. The porous material 614 may be used in any of the condom catheters disclosed herein.

The porous material 614 includes a central region 658 and two or more wings 660 extending from the central region 658. For example, in the illustrated embodiment, the porous material 614 includes four wings 660 extending from the central region 658. The central region 658 and the two or more wings 660 allows the porous material 614 to be formed from a sheet of the porous material 614 without having to sew or glue edges of the sheet together, unlike the porous materials illustrated in FIGS. 4 and 5. As such, the porous material 614 may be easier to form than at least some other porous materials. In an embodiment, the width of the wings 660 may increase with increasing distance from the central region 658. The increased width of the wings 660 allows there to be overlap between the wings 660.

In an embodiment, during use, the porous material 614 may be positioned such that the urethral opening and other portions of the glans of the penis is disposed adjacent to the central region 658 and then the wings 660 may be folded down along a remainder of the penis that is not covered by the central region 658. As such, the central region 658 is able to initially receive and blunt the bodily fluids discharged from the urethral opening and the wings 660 may receive any bodily fluids that may not be received into the central region 658 and instead flow along the shaft of the penis. In an embodiment, the porous material 614 may be positioned in a fluid impermeable layer (not shown) before disposing the porous material 614 on the penis with the central region 658 disposed in the portions of the chamber defined by the end region.

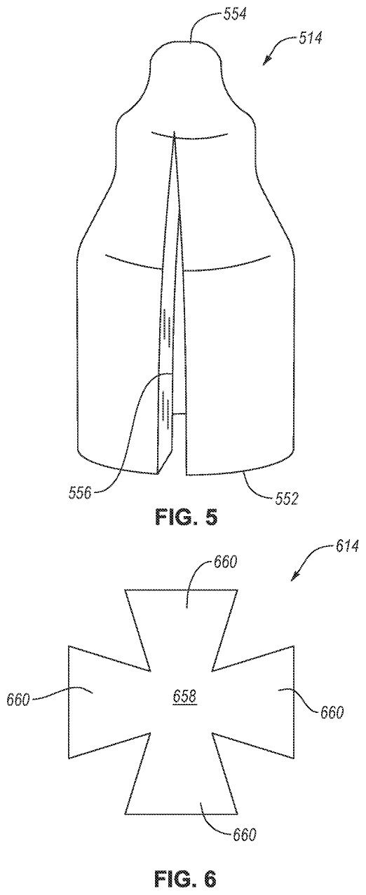

The condom catheters disclosed above use the side wall thereof to attach the condom catheter to the penis and/or to the suprapubic region. However, any of the condom catheters disclosed herein may include one or more attachment structures instead of or in addition to the side wall to attach the condom catheter to the individual. FIG. 7 is a cross-sectional view of a condom catheter 700, according to an embodiment. Except as otherwise disclosed herein. The condom catheter 700 is the same as or substantially similar to any of the condom catheters disclosed herein. For example, the condom catheter 700 includes a fluid impermeable layer 702 including an end region 704 and at least one side wall 706. The condom catheter 700 also includes at least one porous material 514.

The condom catheter 700 includes an attachment structure 762. The attachment structure 762 includes a structure of the condom catheter 700 other than the side wall 706 that is configured to be attached to the region about the penis, such as the mons pubis, the testes, etc. The attachment structure 762 may include an adhesive 734 that is configured to be attached to the region about the penis. In an embodiment, the attachment structure 762 may be integrally formed with the side wall 706. In such an embodiment, the attachment structure 762 is distinguishable from the side wall 706 because the attachment structure 762 flares outwardly relatively to the rest of the side wall 706. For example, the side wall 706 may exhibit a generally cylindrical shape (e.g., is parallel to a longitudinal axis 732 of the condom catheter 700) while the attachment structure 762 extends perpendicular to or obliquely (e.g., exhibit a smallest angle of 30° to 89°) relative to the longitudinal axis 732. The flaring shape of the attachment structure 762 allows the attachment structure 762 to correspond to the region about the penis without needing to be bent, stretched, or otherwise deformed as much as the side wall 706 to exhibit a shape that corresponds to the shape of the region about the penis since such bending, stretching, etc. may uncomfortably pull on the region about the penis.

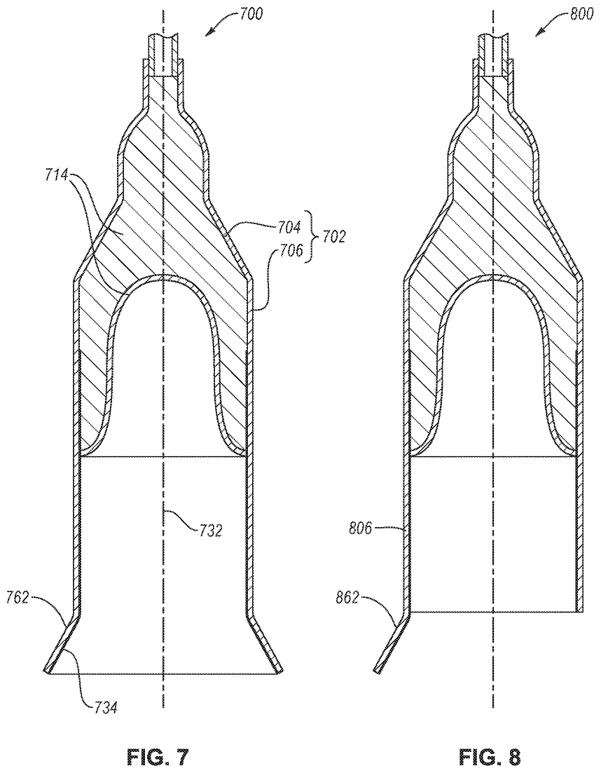

The attachment structure 762 extends around and from the entire circumference of the side wall 706 which inhibits the bodily fluids leaking through the attachment structure 762. However, attaching the attachment structure 762 to the testes or other sensitive anatomy may make removing the condom catheter 700 from the penis especially painful. As such, in some embodiments, the attachment structures disclosed herein may only extend from one or more portions of the side wall such that the attachment structure is only attached to less sensitive regions about the penis (e.g., the mons pubis). For example, FIG. 8 is a cross-sectional view of a condom catheter 800 that includes an attachment structure 862 configured to make removal of the condom catheter 800 less painful, according to an embodiment. The condom catheter 800 is substantially the same as the condom catheter 700 except that the attachment structure 862 only extends from one or more distinct portions of the side wall 806 thereby making removal of the condom catheter 800 less painful than the condom catheter 700 of FIG. 7.

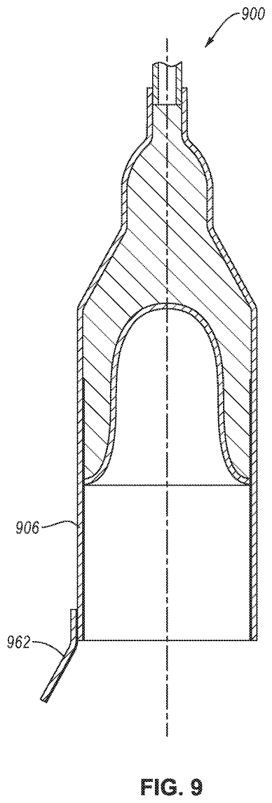

The attachment structure illustrated in FIGS. 7 and 8 are illustrated as being integrally formed with the side walls. However, it is noted that any of the attachments structures disclosed herein may be distinct from the side walls. For example, FIG. 9 is a cross-sectional view of a condom catheter 900 that includes at least one attachment structure 962 that is distinct from the side wall 906, according to an embodiment. Except as otherwise disclosed herein, the condom catheter 900 is the same as or substantially similar to the condom catheters 700 and/or 800.

As previously discussed, the condom catheter 900 includes an attachment structure 962 that is distinct from the side wall 906. The attachment structure 962 that is distinct from the side wall 906 allows the attachment structure 962 to be specially configured for use with the individual. In an example, configuring the attachment structure 962 to be distinct from the side wall 906 allows a user (e.g., medical practitioner) to select an attachment structure 962 exhibiting a shape that corresponds to the shape of the region about the penis. In an example, configuring the attachment structure 962 to be distinct from the side wall 906 allows the attachment structure 962 to exhibit a size that corresponds to the size of the less sensitive anatomy of the individual. In an example, configuring the attachment structure 962 to be distinct from the side wall 906 allows the attachment structure 962 to only be used when necessary (e.g., the condom catheter 900 is not remaining secured to the penis without the attachment structure 962).

The attachment structure 962 may be attached to the side wall 906 using any suitable technique. In an example, as illustrated, the attachment structure 962 may be adhesively attached to the side wall 906. In an example, the attachment structure 962 may be attached to the side wall 906 using a hook-and-loop fastener (e.g., Velcro), sewing the attachment structure 962 to the side wall 906, or using any other suitable technique.

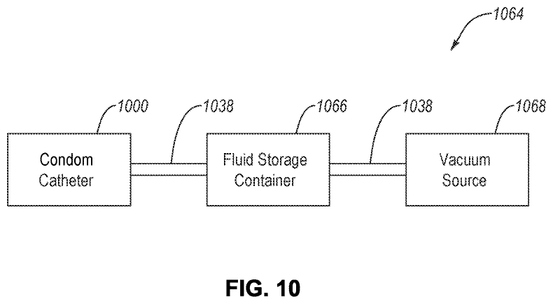

FIG. 10 is a block diagram of a fluid collection system 1064 for fluid collection, according to an embodiment. The fluid collection system 1064 includes a condom catheter 1000, a fluid storage container 1066, and a vacuum source 1068. The condom catheter 1000 may be the same or substantially similar to any of the fluid collection assemblies disclosed herein. The condom catheter 1000, the fluid storage container 1066, and the vacuum source 1068 may be fluidly coupled to each other via one or more conduits 1038. For example, condom catheter 1000 may be operably coupled to one or more of the fluid storage container 1066 or the vacuum source 1068 via the conduit 1038. The bodily fluids collected in the condom catheter 1000 may be removed from the condom catheter 1000 via the conduit 1038 which protrudes into the condom catheter 1000. For example, an inlet of the conduit 1038 may extend into the condom catheter 1000, such as to a reservoir therein. The outlet of the conduit 1038 may extend into the condom catheter 1000 or the vacuum source 1068. Suction force may be introduced into the chamber of the condom catheter 1000 via the inlet of the conduit 1038 responsive to suction (e.g., vacuum) force applied at the outlet of the conduit 1038.

The suction force may be applied to the outlet of the conduit 1038 by the vacuum source 1068 either directly or indirectly. The suction force may be applied indirectly via the fluid storage container 1066. For example, the outlet of the conduit 1038 may be disposed within the fluid storage container 1066 and an additional conduit 1038 may extend from the fluid storage container 1066 to the vacuum source 1068. Accordingly, the vacuum source 1068 may apply suction to the condom catheter 1000 via the fluid storage container 1066. The suction force may be applied directly via the vacuum source 1068. For example, the outlet of the conduit 1038 may be disposed within the vacuum source 1068. An additional conduit 1038 may extend from the vacuum source 1068 to a point outside of the condom catheter 1000, such as to the fluid storage container 1066. In such examples, the vacuum source 1068 may be disposed between the condom catheter 1000 and the fluid storage container 1066.

The fluid storage container 1066 is sized and shaped to retain bodily fluids therein. The fluid storage container 1066 may include a bag (e.g., drainage bag), a bottle or cup (e.g., collection jar), or any other enclosed container for storing bodily fluids such as urine. In some examples, the conduit 1038 may extend from the condom catheter 1000 and attach to the fluid storage container 1066 at a first point therein. An additional conduit 1038 may attach to the fluid storage container 1066 at a second point thereon and may extend and attach to the vacuum source 1068. Accordingly, a vacuum (e.g., suction) may be drawn through condom catheter 1000 via the fluid storage container 1066. Bodily fluids, such as urine, may be drained from the condom catheter 1000 using the vacuum source 1068.

The vacuum source 1068 may include one or more of a manual vacuum pump, an electric vacuum pump, a diaphragm pump, a centrifugal pump, a displacement pump, a magnetically driven pump, a peristaltic pump, or any pump configured to produce a vacuum. The vacuum source 1068 may provide a vacuum or suction to remove bodily fluids from the condom catheter 1000. In some examples, the vacuum source 1068 may be powered by one or more of a power cord (e.g., connected to a power socket), one or more batteries, or even manual power (e.g., a hand operated vacuum pump). In some examples, the vacuum source 1068 may be sized and shaped to fit outside of, on, or within the condom catheter 1000. For example, the vacuum source 1068 may include one or more miniaturized pumps or one or more micro pumps. The vacuum sources 1068 disclosed herein may include one or more of a switch, a button, a plug, a remote, or any other device suitable to activate the vacuum source 1068.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A condom catheter, comprising:
a fluid impermeable layer including:
an end region defining at least a portion of a chamber, the chamber configured to receive at least a portion of a glans of a penis, the end region defining a fluid outlet; and
at least one side wall extending from the end region, the at least one side wall defining an opening configured to receive the penis, the at least one side wall configured to switch between a collapsed state and an expanded state;
at least one porous material configured to be disposed in the chamber, wherein at least a portion of the at least one porous material is not initially disposed in the chamber.

2. The condom catheter of claim 1, wherein the end region defines one or more perforations, the one or more perforations exhibiting a maximum lateral dimension that is greater than about 1 mm.

3. The condom catheter of claim 2, wherein the one or more perforations are formed in the end region adjacent or proximate to the at least one side wall.

4. The condom catheter of claim 1, wherein the end region of the fluid impermeable layer includes a sump extending from the fluid outlet and a bulge region extending from the sump.

5. The condom catheter of claim 4, wherein the at least one porous material occupies or is configured to occupy all of the chamber defined by the sump.

6. The condom catheter of claim 4, wherein the sump includes a generally tapered surface extending from the fluid outlet and a generally cylindrical surface extending from the tapered portion to the bulge region.

7. The condom catheter of claim 4, wherein the bulge region exhibits a generally cylindrical shape.

8. The condom catheter of claim 4, wherein the at least one porous material abuts or is configured to abut substantially all of an interior surface of the bulge region.

9. The condom catheter of claim 1, wherein the at least one side wall is rolled up when the at least one side wall exhibits the collapsed state.

10. The condom catheter of claim 1, wherein at least a portion of an interior surface of the at least one side wall includes an adhesive.

11. The condom catheter of claim 10, wherein substantially all of the interior surface of the at least one side wall includes the adhesive.

12. The condom catheter of claim 1, wherein the fluid impermeable layer further includes a connector extending from the fluid outlet, the connector configured to receive a portion of a conduit.

13. The condom catheter of claim 12, further comprising the conduit in fluid communication with the chamber via the fluid outlet, at least a portion of the conduit disposed in the connector.

14. The condom catheter of claim 12, wherein the at least one porous material occupies or is configured to occupy at least a portion of the connector.

15. The condom catheter of claim 1, wherein the at least one porous material abuts or is configured to abut substantially all of an interior surface of the end region.

16. The condom catheter of claim 1, wherein the at least one porous material abuts or is configured to abut at least a portion of an interior surface of the at least one side wall.

17. The condom catheter of claim 1, wherein the at least one porous material includes a fluid permeable inner layer and a fluid permeable outer layer, the fluid permeable inner layer disposed between at least a portion of the fluid permeable outer layer and at least a portion of the fluid impermeable layer.

18. The condom catheter of claim 17, wherein the at least one fluid permeable outer layer extends around a terminal edge of the fluid permeable inner layer to the fluid impermeable layer, wherein the terminal edge of the fluid permeable inner layer is an edge of the fluid permeable inner layer that is at least one of furthest from the fluid outlet or closest to the opening.

19. The condom catheter of claim 1, wherein a portion of the at least one porous material is rolled up when the at least one side wall exhibits the collapsed state, and wherein a rolled up portion of the at least one porous material is not initially attached to the at least one side wall and is not disposed in the chamber.

20. The condom catheter of claim 1, wherein the at least one porous material is not initially disposed in the chamber or attached to the fluid impermeable layer.

21. The condom catheter of claim 1, wherein the at least one porous material defines a slit.

22. The condom catheter of claim 1, further comprising one or more attachment structures integrally formed with or attached to the side wall, the one or more attachment structures configured to be attached to regions about the penis.

23. A fluid collection system, comprising:
a condom catheter of including:
a fluid impermeable layer including:
an end region defining at least a portion of a chamber, the chamber configured to receive at least a portion of a glans of a penis, the end region defining a fluid outlet; and
at least one side wall extending from the end region, the at least one side wall defining an opening configured to receive the penis, the at least one side wall configured to switch between a collapsed state and an expanded state;
at least one porous material configured to be disposed in the chamber, wherein at least a portion of the at least one porous material is not initially disposed in the chamber;
a fluid storage container; and
a vacuum source;
wherein the chamber of the condom catheter, the fluid storage container, and the vacuum source are in fluid communication with each that, when one or more bodily fluids are present in the chamber, a suction provided from the vacuum source to the chamber of the condom catheter removes the one or more bodily fluids from the chamber and deposits the bodily fluids in the fluid storage container.

24. A method to use a condom catheter, the method comprising:
positioning at least one porous material around at least a portion of a glans of a penis; and
positioning a fluid impermeable layer around the at least a portion of the glans of the penis such that the at least one porous material is positioned between at least a portion of the fluid impermeable layer and the penis, the fluid impermeable layer including:
an end region defining at least a portion of the chamber, the chamber configured to receive at least a portion of the glans of a penis, the end region defining a fluid outlet; and
at least one side wall extending from the end region, the at least one side wall defining an opening configured to receive the penis, the at least one side wall configured to switch between a collapsed state and an expanded state;
wherein the at least one porous material is disposed in the chamber at least after positioning the fluid impermeable layer around the at least a portion of the glans of the glans of the penis; and
wherein at least a portion of the at least one porous material is not initially disposed in the chamber.

25. The method of claim 24, wherein positioning the fluid impermeable layer around the at least a portion of the glans of the penis is performed after positioning the at least one porous material around the at least a portion of the glans of the penis.

26. The method of claim 24, wherein positioning the fluid impermeable layer around the at least a portion of the glans of the penis is performed substantially simultaneously with positioning the at least one porous material around the at least a portion of the glans of the penis.

27. The condom catheter of claim 1, wherein the at least one porous material is not disposed adjacent to at least portions of the fluid impermeable barrier defining the opening when the at least one side wall and the at least one porous material are in the expanded configuration.
